(19) United States
(12) Patent Application Publication
(10) Pub. No.: US 2005/0267556 A1
(43) Pub. Date: Dec. 1, 2005

(54) Drug eluting implants to prevent cardiac apoptosis

(76) Inventors: Allan Shuros, St. Paul, MN (US); Avram Scheiner, Vadnais Heights, MN (US); Rodney Salo, Fridley, MN (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/856,201

(22) Filed: May 28, 2004

(51) Int. Cl. A61N 1/05; A61N 1/362; A61K 9/22

(52) U.S. Cl. 607/120; 607/3; 607/9; 604/891.1

(57) ABSTRACT

Implantable devices are configured to be positioned in or near the heart and to carry and deliver an anti-apoptotic drug to a treatment site in or near the heart. The implantable devices include, but are not limited to, leads, stents, heart valves, atrial septal defect devices, cardiac patches and ventricular restraint devices. Depending on the composition of the device, the drug may be carried by the device through a coating applied to the device, or may be included in the device during the device manufacturing process. The drug may also be included in microparticles, such a microspheres, that are delivered locally through a conduit, such as a catheter.

### BACKGROUND OF THE INVENTION

1. Field of the Invention

The invention relates generally to devices and method for preventing cardiac apoptosis and, more particularly to devices and methods for locally administering anti-apoptotic drugs or agents to reduce apoptosis in cardiac tissue.

2. Description of Related Art

Apoptosis, i.e., programmed cell death, is a normal mechanism leading to cell death and loss for many different organs and tissue. In the heart, apoptosis is believed to contribute significantly to myocardial cell death during and after myocardial infarction. A myocardial infarction is the irreversible damage done to a segment of heart muscle by ischemia, i.e., a decrease in blood supply to an organ due to constriction or obstruction of blood vessels. Apoptosis may also contribute to the development and progression of heart failure after myocardial infarction.

After traumatic events, such as ischemia, surgery, device implant or after chronic high wall stress, apoptosis can turn into a pathological mechanism. Resultant cell loss in the heart can lead to ventricular remodeling and the development or worsening of heart failure with poor prognosis.

Systemic injections of apoptosis inhibitors, e.g., inhibition of Caspase 3, have shown promise in attenuating cell death and ventricular remodeling. While inhibiting apoptosis in the heart may be beneficial during a period of trauma, inhibiting apoptosis globally or in other body organs at the same time or for extended period of time may be detrimental to the patient.

Local delivery of therapeutic substances, by contrast, provides a smaller overall dosage that is concentrated at a specific treatment site. Local delivery can produce fewer side effects and achieve more effective results. Therefore, local delivery of anti-apoptotic drugs to those regions of the heart most affected by a myocardial infarction is desirable.

Hence, those skilled in the art have recognized a need for providing implantable devices for carrying and providing local delivery of anti-apoptotic drugs to areas of the heart. The invention fulfills these needs and others.

### SUMMARY OF THE INVENTION

Briefly, and in general terms, the invention is directed to implantable devices that are configured to be positioned in or near the heart and to carry and deliver an anti-apoptotic drug to a treatment site in or near the heart. The implantable devices include, but are not limited to, endocardial leads, stents, heart valves, atrial septal defect devices, cardiac patches and ventricular restraint devices. Depending on the composition of the device, the drug may be carried by the device through a coating applied to the device, or included in the device during the device manufacturing process. The drug may also be included in microparticles, such a microspheres, that are delivered locally through a conduit, such as a catheter.

These and other aspects and advantages of the invention will become apparent from the following detailed description and the accompanying drawings which illustrate by way of example the features of the invention.

### BRIEF DESCRIPTION OF THE DRAWINGS

FIG. 1 illustrates an endocardial lead positioned in a coronary sinus and/or great cardiac vein;

FIG. 2 illustrates one configuration of the distal portion of the lead of FIG. 1 including a coating carrying an anti-apoptotic drug;

FIG. 3 illustrates another configuration of the distal portion of the lead of FIG. 1 including a matrix carrying an anti-apoptotic drug;

FIG. 3a illustrates another configuration of the distal portion of the lead of FIG. 1 including an osmotic pump carrying an anti-apoptotic drug;

FIG. 4 illustrates another configuration of the distal portion of the lead of FIG. 1 including a collar carrying an anti-apoptotic drug;

FIG. 4a illustrates another configuration of the distal portion of the lead of FIG. 1 including an controlled pump having a reservoir carrying an anti-apoptotic drug;

FIG. 4b illustrates another configuration of the distal portion of the lead of FIG. 1 including a drug matrix whose drug delivery is controlled by electrophoresis;

FIG. 5 illustrates a stent carrying an anti-apoptotic drug mounted an expendable member of a conventional catheter assembly;

FIG. 6 illustrates the stent and expendable member of FIG.5 is an expanded state;

FIG. 7 illustrates the stent of FIG. 6 with the expandable member removed;

FIG. 8 illustrates a heart valve carrying an anti-apoptotic drug positioned in an annulus of a heart;

FIG. 9 illustrates a heart valve, like that shown in FIG. 8, having a sewing cuff for securing the valve to the annulus;

FIG. 10 is a schematic cross-section of the valve of FIG. 9 showing the interface between the sewing cuff and the annulus;

FIG. 11 illustrates an atrial septal defect (ASD) device carrying an anti-apoptotic drug positioned in a shunt between the left and right atria of a heart;

FIG. 12 is a side view of the ASD of FIG. 11;

FIG. 13 is a top view of the ASD of FIG. 12;

FIG. 14 illustrates a heart patch carrying an anti-apoptotic drug; and

FIG. 15 illustrates a ventricular restraint device (VRD) carrying an anti-apoptotic drug positioned on the exterior of a heart.

### DETAILED DESCRIPTION OF THE PREFERRED EMBODIMENTS

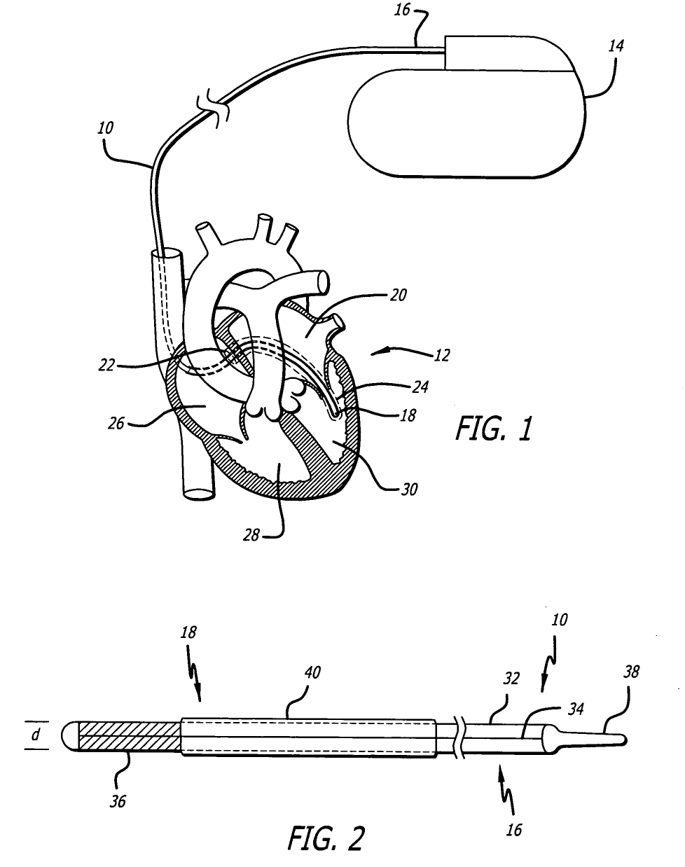

Referring now to the drawings, there are shown in FIGS. 1-15, various implantable devices configured to carry and deliver an anti-apoptotic drug to a local area within a body. Those areas in the body that would benefit from the anti-apoptotic drug include those areas that experience remodeling after infarct and those areas that experiences trauma during surgical implantation of a device.

The implantable devices include, but are not limited to, cardiac rhythm management (CRM) device leads, stents, mechanical heart valves, atrial septal defect (ASD) devices, heart patches and ventricular restraint devices (VRD). Some of these devices, such as leads, patches and VRDs, may be implanted at or near an infarct resulting from an ischemia event. Other devices, such as stents, valves and ASDs, may be implanted in or near the heart in reaction to an ischematic event or other cardiac event or disorder.

Various methods of medicating the implantable devices are described below. While each of these methods is described in association with particular embodiments of the invention, it is understood that the various methods of medicating may be used with other embodiments, depending on the composition of the device. For example, methods of medicating devices having components made of silicone rubber are described in association with CRM device leads. Such methods may, however, be equally applicable to VRDs and cardiac patches. Also, methods of medicating metallic and polymeric components are also described in association with CRM device leads. These methods may find application in other devices such as stents, valves and ASD's.

Leads

With reference to FIG. 1, in one embodiment of the invention, the implantable device is a endocardial lead 10 positioned within a chamber of the heart 12. The lead 10 is part of an implantable CRM device 14 and includes a proximal end 16, which is coupled to the device 14, and a distal end 18, which is coupled on or about one or more portions of the heart 12. A CRM device 14 may be implanted in response to a myocardial infarction and the lead 10 may be positioned in or near an infarct. In other embodiments, the lead may be an epicardial lead.

In FIG. 1, the distal end 18 of the lead 10 is transvenously guided to the left ventricle, through a coronary sinus 22 and into a great cardiac vein 24. This positioning of the lead 10 is useful for delivering pacing and/or defibrillation energy to the left side of the heart 12 such as for treatment of congestive heart failure or other cardiac disorders requiring therapy delivered to the left side of the heart. Other possible positions of the distal portion 18 of the lead 10 include insertion in to the right atrium 26 and/or right ventricle 28, or tranceptal insertion into the left atrium 20 and/or left ventricle 30.

With reference to FIGS. 2, 3 and 4, the distal end region 18 of the lead 10 is configured to carry an anti-apoptotic drug for local delivery to the area around the distal-end region. Thus, only those portions of the implanted lead in proximity to the heart carry the anti-apoptotic drug. Possible anti-apoptotic drugs include: Caspase inhibitors (e.g., of Caspase 3, 8, or 9), Map Kinase inhibitors (e.g., of p38, p43, p53), prevent release of Cytochrome C, apoptosis inducing factor (AIF) inhibitors, tumor necrosis factor (TNF)-α receptor blocker, and Phosphodiesterase inhibitor to increase Ca uptake into sarcoplasmic reticulum. Other drugs may be used in parallel to enhance the effect of the anti-apoptotic drugs including: β adrenergic receptor blockers, angiotensin converting enzyme (ACE) inhibitors, anti-inflammatory drugs and Angiotensin II receptor blockers.

With reference to FIG. 2, in one configuration the endocardial lead 10 includes a biocompatible flexible insulating elongate body 32 (e.g., including a polymer such as medical grade silicone rubber) for translumenal (i.e., transvenous or transarterial) insertion and access within a living organism. In one embodiment, the slender elongate body 32 is tubular and has a peripheral outer surface of diameter d that is small enough for translumenal insertion into the coronary sinus 22 and/or great cardiac vein 24. An elongate electrical conductor 34 is carried within the insulating elongate body 32. The conductor 34 extends substantially along the entire length between the distal end 18 and proximal end 16 of the lead 10, and this length is long enough for the lead 10 to couple the device 14, which is implanted pectorally, abdominally, or elsewhere, to desired locations within the heart 12 for sensing intrinsic electrical heart activity signals or providing pacing/defibrillation-type therapy.

The elongate body 32 forms an insulating sheath covering around the conductor 34. The conductor 34 is coupled to a ring or ring-like electrode 36 at or near the distal end 18 of the elongate body 32. The conductor 34 is coupled to a connector 38 at or near the proximal end 16 of the elongate body 32. The device 14 includes a receptacle for receiving the connector 38, thereby obtaining electrical continuity between the electrode 36 and the device 14.

The electrode 36, or at least a portion thereof, is not covered by the insulating sheath of the elongate body 32. The electrode 36 provides an exposed electrically conductive surface around all, or at least part of, the circumference of the lead 10. In one example, the electrode 36 is a coiled wire electrode that is wound around the circumferential outer surface of the lead 10. The lead 10 also includes other configurations, shapes, and structures of the electrode 36.

The lead 10 includes a biocompatible coating 40 on at least one insulating portion of the peripheral surface of the elongate body 36 at or near the distal end 18. The coating 40 extends circumferentially completely (or at least partially) around the tubular outer peripheral surface of the lead 10 and carries an anti-apoptotic drug. In use, when this lead 10 is inserted and implanted in the body, the coating 40 dissolves and the drug is released. The time duration of the release of the anti-apoptotic drug is preferably between several weeks and months. The time is takes for the coating 40 to fully dissolve and thus for the drug to be completely released may be controlled based on the selection of the coating material and the concentration of the drug.

In one configuration, the coating 40 includes substantially soluble particles dispersed in a substantially insoluble medium, such as biocompatible silicone rubber medical adhesive, other polymer, or other suitable biocompatible adhesive substance. The soluble particles are at least partially dissolvable when exposed to an aqueous substance such as blood or bodily fluids. In accordance with the present invention, the soluble particles include an anti-apoptotic drug. The particles may also include a drug enhancer. When the coating 40 is exposed to an aqueous environment, the substantially soluble drug particles dissolve, providing sustained release of the drug into the surrounding tissue. During manufacture of the lead, one or more portions of the lead is coated with the coating. The coating cures such that it adheres to the lead. Details relating to the coating formation are described in U.S. Pat. No. 6,584,363, titled “Implantable Lead With Dissolvable Coating for Improved Fixation and Extraction,” the disclosure of which is hereby incorporated by reference.

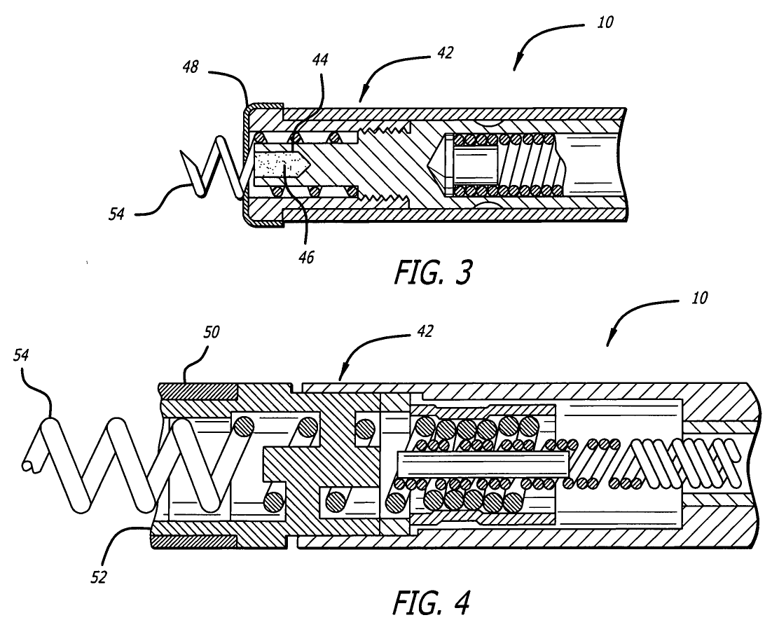

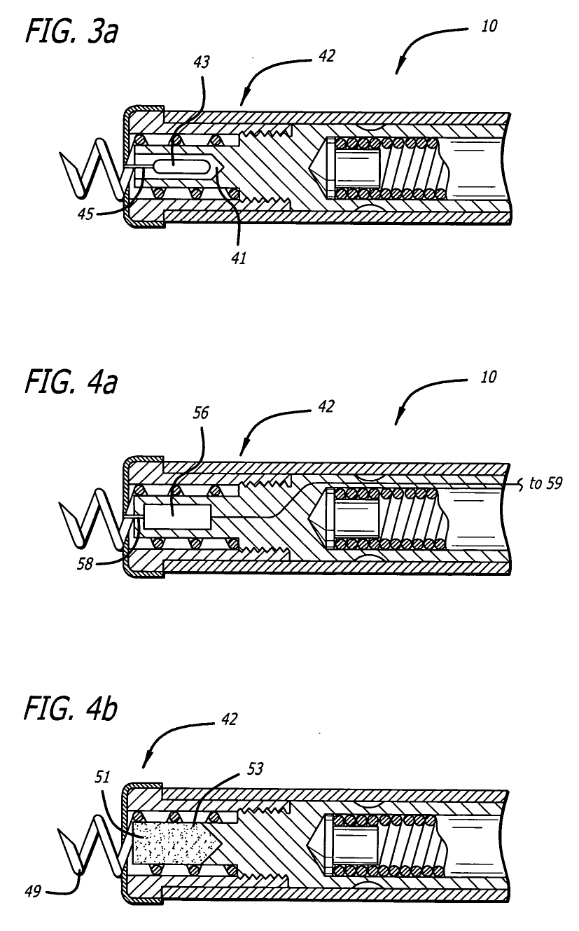

With reference to FIG. 3, in another configuration, the tip 42 of the endocardial lead 10 includes a distal chamber 44 which contains a drug loaded matrix 46. The matrix is preferably a biocompatible silicone adhesive compound impregnated with an anti-apoptotic drug. In use, the 10 lead is inserted and the helix electrode 54 implanted in myocardial tissue. Upon implant, bodily fluid in the vicinity of the selected myocardial location enters the chamber 44 through a screen 48, resulting in the elution of the drug from the matrix 46.

With reference to FIG. 3a, in another configuration, the tip 42 of the lead 10 includes a distal chamber 41 which contains an osmotic pump 43, such as an ALZET osmotic pump (www.alzet.com). The pump 43 includes a reservoir (not shown) filled with an anti-apoptotic drug and an exit port 45 at the tip 42 of the lead. When the lead 10 is positioned in the body, the space in the chamber 41 surrounding the pump 43 is filled with fluid thereby activating operation of the pump. When activated, the fluid in the reservoir is released through the exit port 45.

With reference to FIG. 4, in another configuration, the tip 42 of the endocardial lead 10 includes a drug eluting collar 50. The collar 50 may be a separate element secured to the end of the lead body or may be integrally molded into the distal end of the lead body 52. The collar 50 may take any number of shapes or configurations that may be attached to or otherwise disposed on the distal end of a lead body. The collar 50 is typically in the shape of a ring that is attached over the exterior surface of the lead body 52 or a toroidal insert that is fitted within a cavity at the distal end lead body during manufacture. The collar 50 is generally positioned on the lead body 52 to allow a drug eluted from the collar to come into contact with a target tissue proximate the electrode 54. Thus, the collar 50 is typically secured to the distal end of lead body 52.

To facilitate drug elution, the collar 50 is constructed of a carrier material and an anti-apoptotic drug. The carrier material is typically a silicone rubber or a polymeric matrix, such as polyurethane. Generally, the carrier material is selected and formulated for an ability to incorporate the desired drug during manufacture and release the drug within the patient after implantation. The amount of any particular drug incorporated into collar 50 is determined by the effect desired, the drug potency, the rate at which the drug capacity is released from the carrier material, as well as other factors that will be recognized by those skilled in the art.

A collar 50 in accordance with the present invention may be made by mixing (or dissolving, or melting). The anti-apoptotic drug will typically be mixed with uncured silicone rubber. It may include, but is not necessarily limited to, two part liquid silicone rubbers, gum stock silicone rubbers, or medical adhesives used for creating or bonding silicone rubber components. The drug is added to the uncured silicone rubber in various quantities and following the mixing, the silicone rubber is cured and formed into the collar component for drug delivery. Care should be taken that the method selected does not heat the mixture including the drug beyond a point that would destroy the drug. The collar 50 can be formed by any suitable process, including molding, extruding or other suitable processes recognized by those skilled in the art.

In another configuration, the collar 50 of FIG. 4 may be a microporous collar such as that described in U.S. Pat. No. 6,361,780, titled “Microporous Drug Delivery System,” the disclosure of which is hereby incorporated by reference.

In other configurations, any exposed metallic or polymer component of the lead 10, such as the elongate body 32 (FIG. 2), the electrode 36 (FIG. 2) or the helix electrode 54 (FIG. 3) is coated with the drug. A typical method for coating these components includes applying a composition containing a polymer, a solvent, and a drug to the component using conventional techniques, for example, a dip-coating technique. Dip coating entails submerging all or part of the component or device into a polymer solution.

In another method, a plurality of pores, called “depots,” are formed in the outer surface of the component. The depots are sized and shaped to contain the composition to ensure that a measured dosage of the composition is delivered with the device to the specific treatment site. Depots formed on the components of the implantable device have a particular volume intended to be filled with the composition to increase the amount of the composition that can be delivered from the implantable device to the target treatment site.

The component can be made of a metallic material or an alloy such as, but not limited to, stainless steel, Nitinol, tantalum. nickel-titanium alloy, platinum-iridium alloy, gold, magnesium, or combinations thereof. The component may also be made from bioabsorbable or biostable polymers. A polymeric component should be chemically compatible with any substance to be loaded onto the component.

Depots, which may also be referred to as pores or cavities, can be formed in virtually any component structure at any preselected location. The location of depots within a component varies according to intended usage and application. Depots may be formed on the component by exposing the outer surface to an energy discharge from a laser, such as, but not limited to, an excimer laser. Alternative methods of forming such depots include but are not limited to, physical and chemical etching techniques. Such techniques are well known to one of ordinary skill in the art.

A composition to be applied to the implantable component is prepared by conventional methods wherein all composition components are combined and blended. For example, a predetermined amount of a polymer is added to a predetermined amount of a solvent. The term polymer is intended to include a product of a polymerization reaction inclusive of homopolymers, copolymers, terpolymers, etc., whether natural or synthetic, including random, alternating, block, graft, crosslinked, hydrogels, blends, compositions of blends and variations thereof. The solvent can be any single solvent or a combination of solvents capable of dissolving the polymer. The particular solvent or combination of solvents selected is dependent on factors such as the material from which implantable device is made and the particular polymer selected. Representative examples of suitable solvents include, but are not limited to, aliphatic hydrocarbons, aromatic hydrocarbons, alcohols, ketones, dimethyl sulfoxide (DMSO), tetrahydrofuran (THF), dihydrofuran (DHF), dimethylacetamide (DMAC), acetates and combinations thereof.

Sufficient amounts of a therapeutic substance, e.g., anti-apoptotic drug substance, or a combination of therapeutic substances are then dispersed in the blended composition of the polymer and the solvent. The anti-apoptotic drug substance may be in true solution or saturated in the composition. If the anti-apoptotic drug substance is not completely soluble in the composition, operations such as gentle heating, mixing, stirring, and/or agitation can be employed to effect homogeneity of the residues. However, care should be taken to ensure that the use of heat to effect dissolution does not also cause denaturation of a heat-sensitive anti-apoptotic drug substance.

Alternatively, the anti-apoptotic drug substance may be encapsulated in a sustained delivery vehicle such as, but not limited to, a liposome or an absorbable polymeric particle. The preparation and use of such sustained delivery vehicles are well known to those of ordinary skill in the art. The sustained delivery vehicle containing the anti-apoptotic drug substance is then suspended in the composition.

Inclusion of the anti-apoptotic drug substance in the composition should not adversely alter the composition or characteristic of the anti-apoptotic drug substance. Accordingly, the particular anti-apoptotic drug substance is selected for mutual compatibility with the other components of the composition.

Details of methods of coating or impregnating metallic and/or polymeric components with drugs are described in the following patents which are hereby incorporated by reference: U.S. Pat. No. 6,287,628, titled “Porous Prosthesis and a Method of Depositing Substances into the Pores;” U.S. Pat. No. 6,506,437, titled “Methods of Coating an Implantable Device Having Depots Formed in a Surface Thereof;” U.S. Pat. No. 6,544,582, titled “Method and Apparatus for Coating an Implantable Device;” U.S. Pat. No. 6,555,157, titled “Method for Coating an Implantable Device and System for Performing the Method;” U.S. Pat. No. 6,585,765, titled “Implantable Device Having Substances Impregnated Therein and a Method of Impregnating the Same” and U.S. Pat. No. 6,616,765, titled “Apparatus and Method for Depositing a Coating onto a Surface of a Prosthesis.”

While the various embodiments of the lead configuration of the invention described thus far have been passive delivery devices, active delivery embodiments are contemplated. For example, with reference to FIG. 4a, in one active delivery embodiment, the tip 42 of the lead includes a pump reservoir 56 and delivery tube 58. The reservoir 56 is filled with an anti-apoptotic drug and delivery of the drug through the tube 58 is controlled by a pump controller 59 in the CRM housing. In an alternate configuration of this embodiment, the reservoir 56 may be located in the CRM housing and the delivery tube 58 extends the length of the lead from the tip to the CRM housing.

In another active delivery embodiment, electrophoresis is used to control delivery of the anti-apoptotic drug. With reference to FIG. 4b, in this embodiment a drug matrix 51 configured to release its drug when it is subjected to an electric field is carried in a chamber 53 in the tip 42 of the lead. The matrix 51 is surrounded by a helix tip electrode 49 configured to carry a non-pacing current. This current subjects the drug matrix 51 contained in the chamber 53 to an electric field causing release of the drug from the matrix.

Stents

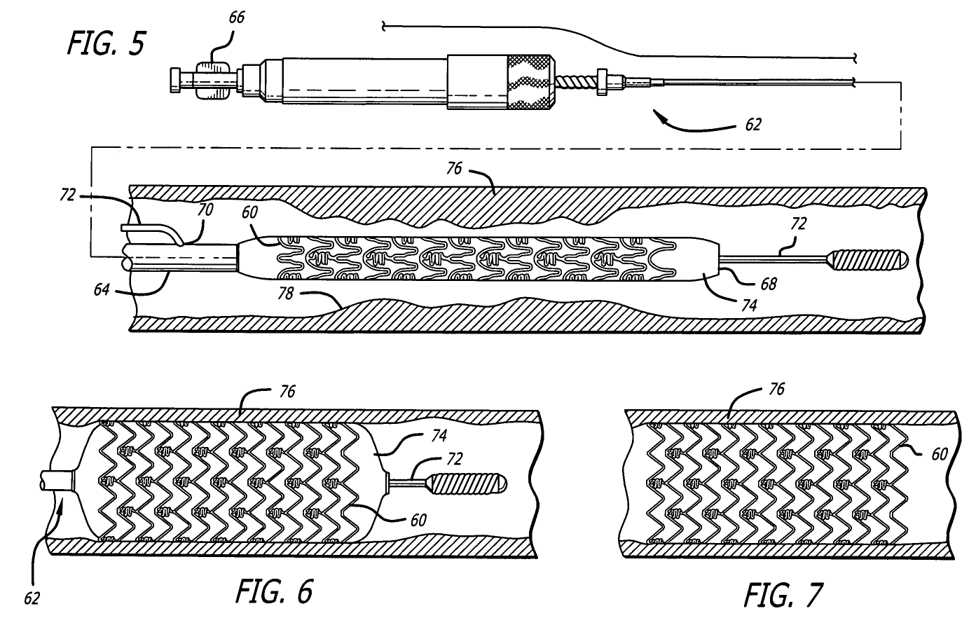

With reference to FIGS. 5, 6 and 7, in another embodiment of the invention, the implantable device is stent 60 positioned within the vascular system. As shown in FIG. 5, a stent 60 is mounted on a conventional catheter assembly 62 which is used to deliver the stent and implant it in a body lumen, such as a coronary artery, peripheral artery, or other vessel or lumen within the body. The catheter assembly includes a catheter shaft 64 which has a proximal end 66 and a distal end 68. The catheter assembly 62 is configured to advance through the patient's vascular system by advancing over a guide wire 72 by any of the well known methods of an over the wire system (not shown) or a well known rapid exchange catheter system.

Catheter assembly 62 as depicted in FIG. 5 is of the well known rapid exchange type which includes an RX port 70 where the guide wire 72 will exit the catheter. The distal end of the guide wire 72 exits the catheter distal end 68 so that the catheter advances along the guide wire on a section of the catheter between the RX port 70 and the catheter distal end 68. As is known in the art, the guide wire lumen which receives the guide wire is sized for receiving various diameter guide wires to suit a particular application. The stent 60 is mounted on the expandable member 74 and is crimped tightly thereon so that the stent and expandable member present a low profile diameter for delivery through the arteries.

As shown in FIG. 5, a partial cross-section of an artery 76 is shown with a small amount of plaque that has been previously treated by an angioplasty or other repair procedure. Stent 60 is used to repair a diseased or damaged arterial wall which may include plaque 78 as shown in FIG. 5, or a dissection, or a flap which are sometimes found in the coronary arteries, peripheral arteries and other vessels.

In a typical procedure to implant the stent 60, the guide wire 72 is advanced through the patient's vascular system by well known methods so that the distal end of the guide wire is advanced past the plaque or diseased area 78. Prior to implanting the stent, the cardiologist may wish to perform an angioplasty procedure or other procedure, i.e., atherectomy, in order to open the vessel and remodel the diseased area. Thereafter, the stent delivery catheter assembly 62 is advanced over the guide wire 72 so that the stent 60 is positioned in the target area. The expandable member or balloon 74 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIG. 6, the balloon 74 is fully inflated with the stent 60 expanded and pressed against the vessel wall, and in FIG. 7, the implanted stent 60 remains in the vessel after the balloon has been deflated and the catheter assembly 62 (FIG. 6) and guide wire 72 have been withdrawn from the patient.

The stent 60 serves to hold open the artery after the catheter is withdrawn, as illustrated by FIG. 7. Due to the formation of the stent from an elongated tubular member, the undulating components of the stent are relatively flat in transverse cross-section. When the stent is expanded, it is pressed into the wall of the artery and accordingly does not interfere with the blood flow through the artery. The stent is pressed into the wall of the artery and will eventually be covered with endothelial cell growth which further minimizes blood flow interference.

In one configuration, the entire surface of the stent 60 is configured to carry and deliver an anti-apoptotic drug. In another configuration, only select surfaces, e.g., the tissue contacting surface, carry the anti-apoptotic drug. The stent 60 may be formed of either a metal or a polymer material and thus the methods available for medicating the stent are the same as those described above with respect to the metallic and polymeric components of the lead configuration.

Heart Valves

Prosthetic heart valves are utilized to replace malformed, damaged, diseased or otherwise malfunctioning valves in body passageways, such as heart valves, including the tricuspid valve, the mitral valve, the aortic valve and the pulmonary valve. Such prosthetic heart valves are typically implanted into the heart either by open chest surgery which requires a sternotomy or by minimally invasive surgery which requires a thoracotomy between adjacent ribs.

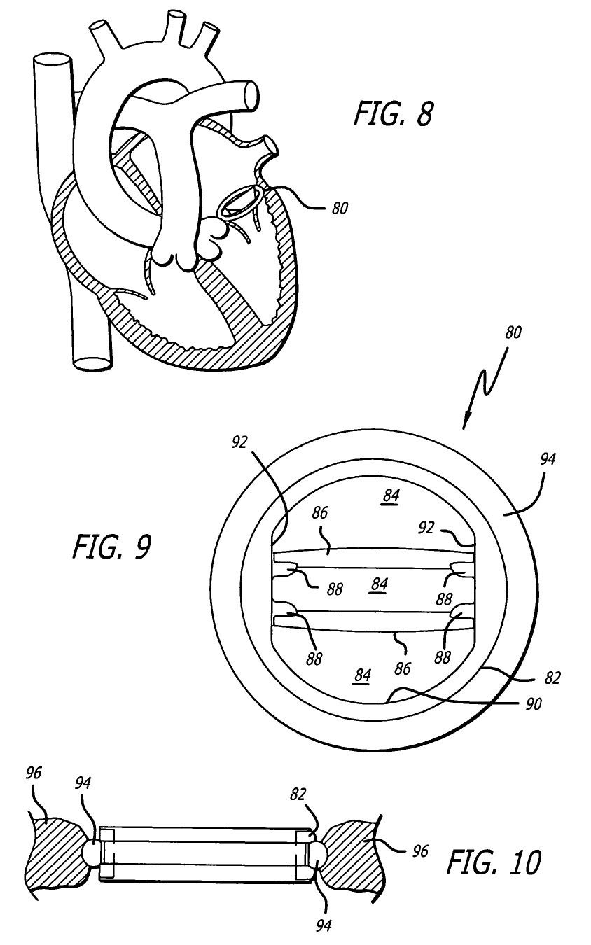

With reference to FIGS. 8, 9 and 10, in another embodiment of the invention, the implantable device is a mechanical heart valve 80 positioned within the heart. As shown in FIG. 9, the heart valve 80 includes a generally circular orifice body or housing 82 having a generally circular orifice or opening 84 extending therethrough. Although illustrated as a circular housing 82 and circular opening 84, those skilled in the art will recognize that many suitable shapes may be employed, depending on the anatomical geometry of the implant site.

The heart valve prosthesis 80 further includes a pair of occluders or leaflets 86 disposed in the opening 84. The leaflets 86 are pivotally mounted to the valve housing 82 and are movable between an open position and a closed position. When the leaflets 86 are in their closed position, the opening 84 is substantially closed. Conversely, when the leaflets 86 are in their open position, the opening 84 is substantially open thus allowing the passage of blood therethrough. Although two leaflets 86 are illustrated in FIG. 9, those skilled in the art will recognize that any suitable number of leaflets may be utilized.

The heart valve prosthesis 80 further includes protrusion stops 84 extending from the lumen surface 90 of the housing 82. The stops 88 are preferably on the downstream edge of housing 82 or a suitable location on flat portion 92. The protrusion stops 84 serve to limit the motion of the leaflets 86 as the leaflets move to their open position. The heart valve 80 also includes a sewing cuff 94 through which the valve is secured to the annulus of the heart.

With reference to FIG. 10, the housing 82 is coupled to a tissue annulus 96 through the sewing cuff 94. The sewing cuff 94 is typically formed of silicone. The sewing cuff 94 is provided as one example of an attachment mechanism for attaching the housing 82 to the tissue annulus 96. Any other type of attachment mechanism as known or contemplated in the art may be utilized, such as helical screws.

A traumatic event such as valve replacement surgery may lead to pathological apoptosis. In order to reduce the possibility of pathological apoptosis, the attachment mechanism 94 of the heart valve 80 is configured to carry an anti-apoptotic drug. In a preferred embodiment, only portions of the valves directly contacting tissue are configured to carry and deliver an anti-apoptotic drug. The actual valve surfaces only contacting blood may not be coated. In other embodiments, all components of the valve may be configured to carry and deliver an anti-apoptotic drug.

With reference to FIG. 9, in one configuration, the entire surface of the sewing cuff 94 is coated with an anti-apoptotic drug in a manner similar to that previously described with respect to the lead coating configuration. Alternatively, the sewing cuff may be formed to include the drug in a manner similar to that previously described with respect to the lead collar configuration. In an alternate configuration, only that portion of the cuff 94 that directly contacts the tissue 96 carries the anti-apoptotic drug. In valve configuration that do not have a cuff and are attached by other means, the drug may be carried by such means and/or the housing 82.

Atrial Septal Defect/Patent Foramen Ovale Devices

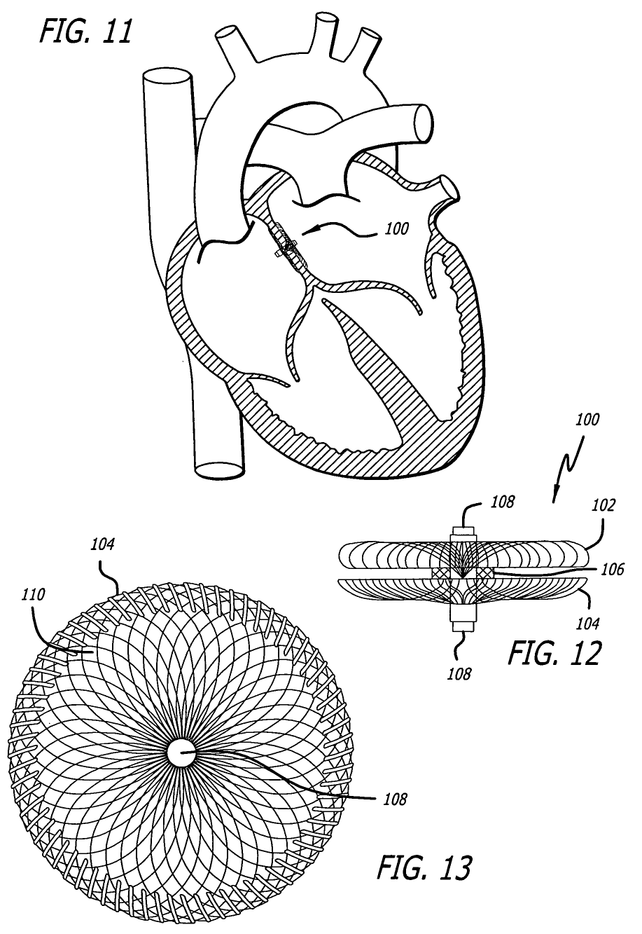

With reference to FIGS. 11, 12 and 13, in another embodiment of the invention, the implantable device is a device for correcting an atrial septal defect (ASD). ASD is a congenital abnormality of the atrial septum characterized by structural deficiency of the atrial septum. A shunt may be present in the atrial septum, allowing flow between the right and left atriums. In large defects with significant left to right shunts through the defect, the right atrium and right ventricle are volume overloaded and the augmented volume is ejected into a low-resistance pulmonary vascular bed. As shown in FIG. 12, the device 100 in its relaxed, unstretched state has two aligned disks 102 and 104 linked together by a short middle cylindrical section 106. This device 100 may also be well suited in occluding defects known in the art as patent foramen ovale (PFO).

Regarding the constructional features of the device 100, the ASD occluder is sized in proportion to the shunt to be occluded. In the relaxed orientation, the occluder, which is formed of metal fabric, is shaped such that two disk members 102 and 104 are axially aligned and linked together by the short cylindrical segment 106. The length of the cylindrical segment 106 preferably approximates the thickness of the atrial septum, and ranges between 2 to 20 mm. The proximal 102 and distal 104 disks preferably have an outer diameter sufficiently larger than the shunt to prevent dislodging of the device.

The ends of the tubular braided metal fabric device 100 are welded or clamped together with clamps 108, similar to those described above to avoid fraying. The clamp 108 tying together the wire strands at one end also serves to connect the device to a delivery system. In the embodiment shown, the clamp 108 is generally cylindrical in shape and has a recess for receiving the ends of the metal fabric to substantially prevent the wires comprising the woven fabric from moving relative to one another. The clamp 108 also has a threaded surface within the recess adapted to receive and engage a threaded distal end of a delivery device.

The ASD occlusion device 100 is preferably made from a 0.005 inch nitinol wire mesh. The braiding of the wire mesh may be carried out with 28 picks per inch at a shield angle of about 64 degrees using a Maypole braider with 72 wire carriers. The stiffness of the ASD device 100 may be increased or decreased by altering the wire size, the shield angle, the pick size, the number of wire carriers or the heat treatment process. The ASD device 100 includes an occluding fabric 110 of known suitable construction contained within the interior of the device.

The ASD device 100 is configured to carry an anti-apoptotic drug. In a preferred embodiment, all portions of the ASD device 100 that would be permanently implanted in the body are coated with the anti-apoptotic drug. The ASD device 100 is typically formed of a metal and thus the methods available for medicating the ASD device are the same as those described above with respect to the metallic components of the lead configuration. Alternatively, if the ASD device is formed of a polymer it may be medicated in the same way as the metallic form. Furthermore, either the metallic or the polymeric form may be coated with a medicated silicone rubber coating similar to that described above with respect to the lead coating configuration.

Cardiac Patches

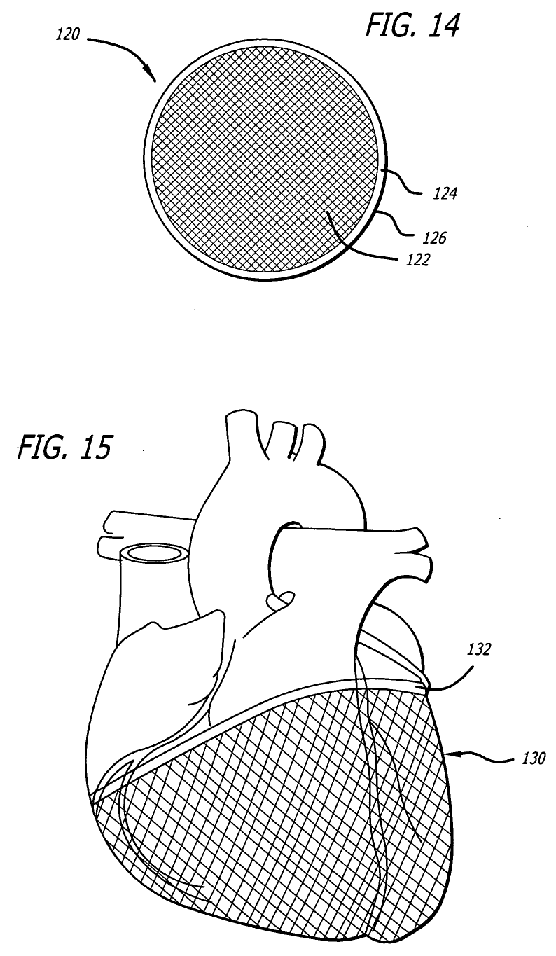

With reference to FIG. 14, in another embodiment of the invention the implantable device is a cardiac patch 120. The patch 120 provides reinforcement of the heart wall at a localized area, such as a cardiac aneurysm or at an area of the myocardium which has been damaged due to myocardial infarction. The patch 120 includes a mesh biomedical material 122 having a thickened peripheral ring 124 which reinforces the peripheral edge 126 of the patch for attachment of the patch to the epicardial surface of the heart.

The patch 120 can be applied to the epicardial surface of the heart over or under the parietal pericardium. The patch 120 is typically applied to the epicardial surface by suturing around the periphery 126 of the patch. Generally, a patch is applied to the epicardium through a thoracotomy or other incision providing sufficient exposure of the heart.

The patch 120 is made from biomedical, and preferably biodegradable, material which can be applied to the epicardial surface of the heart. Examples of suitable biomedical materials include perforate and non-perforate materials. Perforate materials include, for example, a mesh such as a polypropylene or polyester mesh. Non-perforate materials include, for example, silicone rubber. In a preferred embodiment, the patch is an open mesh material.

The patch 120 is configured to carry and deliver an anti-apoptotic drug. Depending on its composition, the patch may either be coated with the anti-apoptotic drug, such as a silicone rubber coating as previously described with respect to the lead coating configuration, or the drug may be included during the patch manufacturing process in a manner similar to that described with respect to the lead collar configuration.

Ventricular Restraint Devices

With reference to FIG. 15, in another embodiment of the invention the implantable device is a ventricular restraint device (VRD) 130. Such a device 130 is placed around and attached to the heart to support damaged heart muscles and to limit outward expansion of the heart wall. When applied to the heart, a VRD 130 can be placed over or under the parietal pericardium. The VRD 130 can be secured to the epicardium by a securing arrangement mounted at the base of the jacket. A suitable securing arrangement includes, for example, a circumferential attachment device 132, such as a cord, suture band, adhesive or shape memory element which passes around the circumference of the base of the VRD 130. The ends of the attachment device 132 can be fastened together to secure the VRD 130 in place. Alternatively, the base of the VRD 130 can be reinforced for suturing the base of the jacket to the epicardium

A VRD 130 is made from a biomedical material which can be applied to the epicardial surface of the heart. A VRD 130 can be prepared from an elastic or substantially non-elastic biomedical material. The biomedical material can be inflexible, but is preferably sufficiently flexible to move with the expansion and contraction of the heart without impairing systolic function. The biomedical material should, however, constrain cardiac expansion, during diastolic filling of the heart, to a predetermined size. Examples of suitable biomedical materials include perforate and non-perforate materials. Perforate materials include, for example, a mesh such as a polypropylene or polyester mesh. Non-perforate materials include, for example, silicone rubber or an open-pore foam, such as silicone foam.

The VRD 130 is configured to carry and deliver an anti-apoptotic drug. Like the cardiac patch, depending on its composition, the VRD 130 may either be coated with the anti-apoptotic drug, such as a silicone rubber coating as previously described with respect to the lead coating configuration, or the drug may be included during the VRD manufacturing process in a manner similar to that described with respect to the lead collar configuration. All permanently implanted components of the VRD may be coated with the antiapoptotic drug. This prevents immediate acute tissue death due to device/tissue interaction and inhibits chronic cell death due to the heart failure. In a preferred embodiment the entire attachment device 132 and the surface of the mesh facing the heart tissue are coated.

Microparticles

In another embodiment of the invention, local delivery of an anti-apoptotic drug is achieved using a microparticle, polymeric matrix delivery system which releases the drug into surrounding tissue. Both non-biodegradable and biodegradable matrices can be used for delivery of the drug, although biodegradable matrices are preferred. These may be natural or synthetic polymers, although synthetic polymers are preferred due to the better characterization of degradation and release profiles. The polymer is selected based on the period over which drug release is desired. The microparticles can be microspheres, where the drug is dispersed within a solid polymeric matrix, or microcapsules, where the core is of a different material than the polymeric shell, and the drug is dispersed or suspended in the core, which may be liquid or solid in nature.

Bioerodible microspheres can be prepared using any of the methods developed for making microspheres for drug delivery, for example, as described by Mathiowitz and Langer, J. Controlled Release 5,13-22 (1987); Mathiowitz, et al., Reactive Polymers 6, 275-283 (1987); and Mathiowitz, et al., J. Appl. Polymer Sci. 35, 755-774 (1988), the teachings of which are hereby incorporated by reference. The selection of the method depends on the polymer selection, the size, external morphology, and crystallinity that is desired, as described, for example, by Mathiowitz, et al., Scanning Microscopy 4,329-340 (1990); Mathiowitz, et al., J. Appl. Polymer Sci. 45, 125-134 (1992); and Benita, et al., J. Pharm. Sci. 73, 1721-1724 (1984), the teachings of which are incorporated herein.

Delivery of the microspheres is facilitated by a catheter placed in or near the treatment site. The tip of the catheter is placed upstream from the target treatment site such that when the microspheres are released through the catheter tip, they disperse and lodge themselves in the treatment area, e.g., wall of a vein or artery.

It will be apparent from the foregoing that while particular forms of the invention have been illustrated and described, various modifications can be made without departing from the spirit and scope of the invention. Accordingly, it is not intended that the invention be limited, except as by the appended claims.

1. An implantable device configured to be positioned in or near the heart and to carry and deliver an anti-apoptotic drug to a treatment site.

2. The device of claim 1 wherein the anti-apoptotic drug comprises at least one of Caspase inhibitors, Map Kinase inhibitors, AIF inhibitors, TNF-α receptor blocker and Phosphodiesterase inhibitor.

3. The device of claim 1 wherein the anti-apoptotic drug is configured to prevent the release of Cytochrome C.

4. The device of claim 1 wherein the anti-apoptotic drug further comprises at least one of β adrenergic receptor blockers, ACE inhibitors, anti-inflammatory drugs and Angiotensin II receptor blockers.

5. The device of claim 1 wherein the implantable device comprises a lead and the anti-apoptotic drug is included in a coating applied to the lead.

6. The device of claim 1 wherein the implantable device comprises a lead and the drug is included in a drug eluting matrix carried by the lead.

7. The device of claim 6 wherein the drug eluting matrix is configured to elute the drug upon contact with fluid

8. The device of claim 6 wherein the drug eluting matrix is at least partially positioned to be subjected to an electric field and is configured to elute the drug when subjected to the electric field.

9. The device of claim 1 wherein the implantable device comprises a lead and the anti-apoptotic drug is included in a silicone based component secured to the lead.

10. The device of claim 1 wherein the implantable device comprises a lead and the anti-apoptotic drug is included in an osmotic pump carried by the lead.

11. The device of claim 1 wherein the implantable device comprises a lead and the anti-apoptotic drug is included in a controllable pump carried by the lead.

12. The device of claim 1 wherein the implantable device comprises a stent and the anti-apoptotic drug is included in a coating applied to the stent.

13. The device of claim 1 wherein the implantable device comprises a heart valve and the anti-apoptotic drug is included in a coating applied to a component of the valve.

14. The device of claim 1 wherein the implantable device comprises a heart valve and the anti-apoptotic drug is included in a component of the valve.

15. The device of claim 1 wherein the implantable device comprises an anterior septal defect device or a patent foramen ovale device and the anti-apoptotic drug is included in a coating applied to a component of the device.

16. The device of claim 1 wherein the implantable device comprises a cardiac patch and the anti-apoptotic drug is included in a coating applied to a component of the patch.

17. The device of claim 1 wherein the implantable device comprises a cardiac patch and the anti-apoptotic drug is included in a component of the patch.

18. The device of claim 1 wherein the implantable device comprises a ventricular restraint device and the anti-apoptotic drug is included in a coating applied to a component of the device.

19. The device of claim 1 wherein the implantable device comprises a ventricular restraint device and the anti-apoptotic drug is included in a component of the device.

20. A system for providing local delivery of an anti-apoptotic drug to a local delivery area, said device comprising:
an implantable drug delivery conduit adapted to be positioned at or near the local delivery area;
a reservoir of microparticles carrying an anti-apoptotic drug, the reservoir in fluid communication with the drug delivery conduit; and
a pump for transferring the microparticles from the reservoir into the conduit for delivery at the local delivery area.

21. The system of claim 20 wherein the microparticles comprise microspheres.

22. A method of treating an ischemic or infarcted region of a heart comprising:
medicating an implantable device with an anti-apoptotic drug; and
positioning the implantable device at or near the infarcted region.

23. The method of claim 22 wherein medicating comprises applying a coating carrying the drug to a component of the implantable device.

24. The method of claim 22 wherein medicating comprises manufacturing a component of the implantable device to include the drug.
